(19) United States
(12) Patent Application Publication
(10) Pub. No.: US 2026/0131067 A1
(43) Pub. Date: May 14, 2026

(54) SURGICAL CASSETTE TUBING ADAPTER AND SENSING FEATURES

(71) Applicant: Alcon Inc., Fribourg (CH)

(72) Inventors: Yin Hua Chen, Mission Viejo, CA (US); Raphael Gordon, Ladera Ranch, CA (US); Behzad Hashemi, Irvine, CA (US)

(21) Appl. No.: 19/364,361

(22) Filed: Oct. 21, 2025

Related U.S. Application Data
(60) Provisional application No. 63/720,256, filed on Nov. 14, 2024.

(51) Int. Cl. A61M 1/00; A61M 39/10; A61F 9/007

(52) U.S. Cl. CPC A61M 1/772; A61M 1/72; A61M 1/743; A61M 39/10; A61F 9/007; A61M 2039/1077; A61M 2205/14; A61M 2205/3313; A61M 2210/0612

(57) ABSTRACT

In certain embodiments, a surgical cassette for ophthalmic surgical procedures includes a base and a tubing adapter coupled to the base. At least one pump assembly and one or more valve assemblies are disposed in the base. The base further includes a plurality of first channels in fluid communication with the at least one pump assembly. The surgical cassette may further include one or more sensing regions for facilitating detection of the surgical cassette by a surgical console. The one or more valve assemblies are configured to control fluid communication between the plurality of first channels of the base. The tubing adapter includes a plurality of ports in fluid communication with at least one of the plurality of first channels disposed in the base, wherein each of the plurality of ports is configured to receive a corresponding fluidic tube for coupling the fluidic tube to the surgical cassette.

### INTRODUCTION

Ophthalmic surgical procedures are often classified as anterior segment surgical procedures, posterior segment procedures, or combined anterior segment and posterior segment procedures (i.e., “combined procedures”). The anterior segment refers to the front-most region of the eye, and includes the cornea, iris, and lens. Thus, anterior segment surgical procedures typically include surgeries performed on the iris and/or lens, such as cataract surgery. The posterior segment refers to the back-most region of the eye that includes the anterior hyaloid membrane and the optical structures behind it, such as the vitreous humor, the retina, the choroid, and the optic nerve. Posterior segment surgical procedures typically include retinal and vitreoretinal surgeries. In certain cases, a patient may have pathologies of the eye requiring both anterior and posterior procedures; in such cases, a combined procedure may be performed.

During anterior and/or posterior segment surgery, tissue fragments and other materials may be aspirated or suctioned out of the eye through, e.g., a hollow needle or cannula, and into a drain reservoir or other waste receptacle. Also, during the procedure, an irrigating or infusion fluid may be pumped into the eye to maintain an intraocular pressure (IOP) and prevent collapse of the eye. A surgical cassette having one or more peristaltic and/or venturi pumps and one or more valve assemblies may be operably coupled with a fluidics module of a surgical console and used to facilitate the aspiration/suction and irrigation/infusion functionalities described above. In general, the one or more valve assemblies of the surgical cassette are operable to control the application of pressure and vacuum generated by the one or more peristaltic pumps during the surgical procedure. Fluids are delivered to and/or removed from the surgical site via flexible tubing extending between the surgical cassette and one or more surgical tools utilized at the surgical site.

Yet, technological advances in surgical cassette design have brought with them the utilization of new materials, which in turn, introduces a new set of technical challenges such as, the challenge of maintaining a robust hermetic seal with the certain types of tubing utilized to convey fluids to and/or from surgical sites, as well as the challenge of facilitating consistent detection of attachment of the surgical cassettes to surgical consoles.

Therefore, there is a need for improved surgical cassettes which address at least some of the drawbacks described above.

### BRIEF SUMMARY

The present disclosure relates generally to ophthalmic surgical cassettes and methods of use thereof.

In certain embodiments, a surgical cassette is provided for ophthalmic surgical procedures, the surgical cassette comprising: a base, comprising: at least one pump assembly disposed in the base; a plurality of channels in fluid communication with the at least one pump assembly, wherein one or more of the channels is in fluid communication with a source of pressure or vacuum; and one or more valve assemblies disposed in the base and configured to control fluid communication between the plurality of channels of the base; and a tubing adapter coupled to the base, the tubing adapter comprising a plurality of ports in fluid communication with at least one of the plurality of channels disposed in the base, each of the plurality of ports configured to receive a corresponding fluidic tube for coupling the fluidic tube to the surgical cassette.

In certain embodiments, a surgical cassette is provided for ophthalmic surgical procedures, the surgical cassette comprising: a base, comprising: at least one pump assembly disposed in the base; a plurality of first channels in fluid communication with the at least one pump assembly, wherein one or more of the first channels is in fluid communication with a source of pressure or vacuum; one or more valve assemblies disposed in the base and configured to control fluid communication between the plurality of channels of the base; and one or more sensing regions disposed on an external surface of the base for facilitating detection of the surgical cassette when the surgical cassette is attached to a surgical console; and a cover coupled to the base, the cover comprising at least one port in fluid communication with at least one of the plurality of channels disposed in the base.

The following description and the related drawings set forth in detail certain illustrative features of one or more embodiments.

### BRIEF DESCRIPTION OF THE DRAWINGS

The appended figures depict certain aspects of one or more disclosed embodiments and are therefore not to be considered limiting of the scope of this disclosure.

FIG. 1A illustrates an example of an ophthalmic surgical system that may be used to perform ophthalmic procedures on an eye, according to certain embodiments.

FIG. 1B is an example of subsystems of a console of the ophthalmic surgical system of FIG. 1A, according to certain embodiments.

FIGS. 2A-2E are various views of an example surgical cassette which may be operably coupled to a console of the ophthalmic surgical system of FIG. 1A, according to certain embodiments. FIG. 2A is a back plan view of the surgical cassette; FIG. 2B is a side elevation view of the surgical cassette; FIG. 2C is a front plan view of the surgical cassette; FIG. 2D is bottom-up elevation view of the surgical cassette; and FIG. 2E is a perspective exploded view of the surgical cassette.

FIGS. 3A-3C are various views of a portion of the example surgical cassette of FIGS. 2A-2E, according to certain embodiments. FIG. 3A is an enlarged perspective view of the portion of the surgical cassette; FIG. 3B is a back plan view of the portion of the surgical cassette; and FIG. 3C is a cross-sectional view of the portion of the surgical cassette.

FIGS. 4A-4E are various views of a portion of the example surgical cassette of FIGS. 2A-2E, according to certain embodiments. FIG. 4A is an enlarged perspective view of the portion of the surgical cassette; FIG. 4B is another enlarged perspective view of the portion of the surgical cassette; FIG. 4C is a back plan view of the portion of the surgical cassette; FIG. 4D is an elevated view of the portion of the surgical cassette; and FIG. 4E is a cross-sectional view of the tubing adapter 210.

FIGS. 5A and 5B are various views of the surgical cassette of FIGS. 2A-2E, including a tubing adapter, attached to fluidic tubes, according to certain embodiments. FIG. 5A is a perspective view of the surgical cassette attached to the fluidic tubes; FIG. 5B is an enlarged view of a portion of the surgical cassette, including the tubing adapter, attached to the fluidic tubes.

FIGS. 6A and 6B are various views of an example surgical cassette which may be operably coupled to a console of the ophthalmic surgical system of FIG. 1A, according to certain embodiments. FIG. 6A is a back plan view of the surgical cassette; and FIG. 6B is an elevation view of the surgical cassette.

FIGS. 7A and 7B are various views of the cassette bay of the surgical console of FIG. 1A, without and with a surgical cassette attached thereto, according to certain embodiments. FIG. 7A is an enlarged plan view of the cassette bay without a surgical cassette attached thereto; FIG. 7B is an enlarged plan view of the cassette bay with a surgical cassette attached thereto.

To facilitate understanding, identical reference numerals have been used, where possible, to designate identical elements that are common to the drawings. It is contemplated that elements and features of one embodiment may be beneficially incorporated in other embodiments without further recitation.

### DETAILED DESCRIPTION

The present disclosure relates generally to ophthalmic surgical cassettes and methods of use thereof.

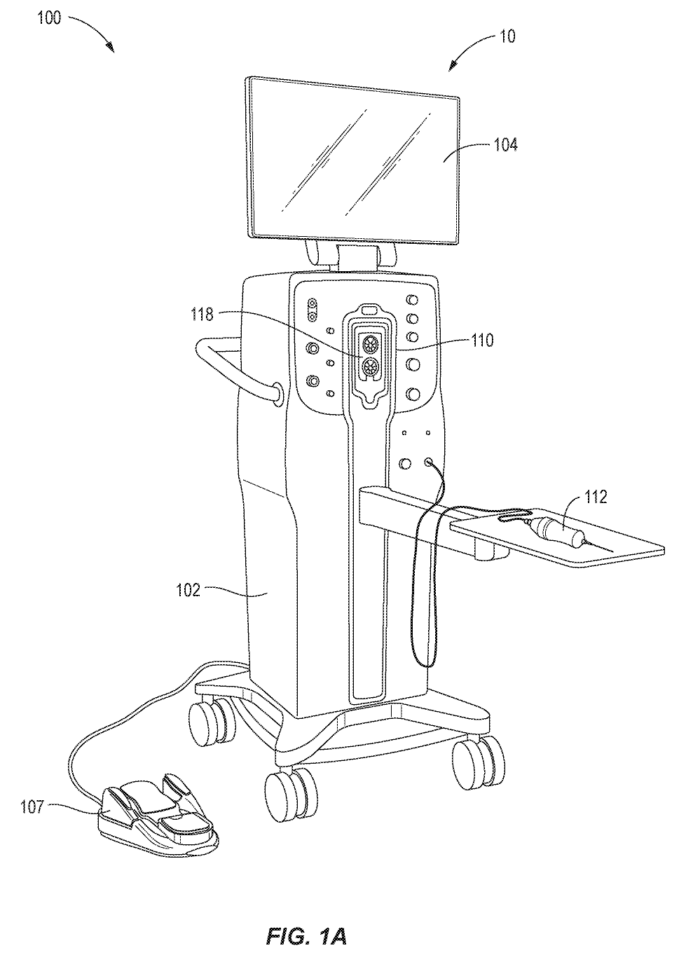

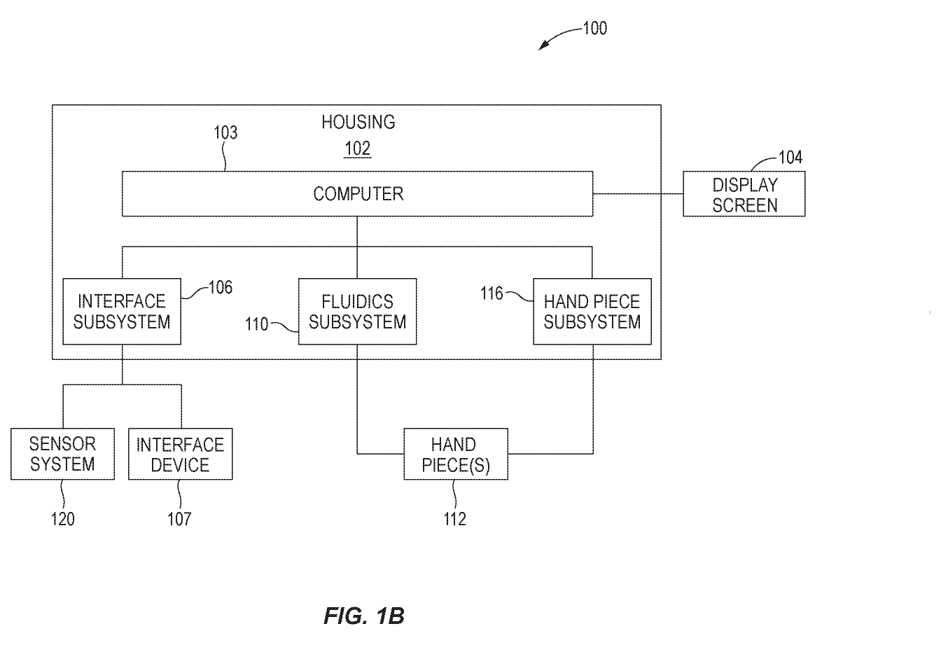

FIG. 1A illustrates an example of an ophthalmic surgical system 10 that may be used to perform ophthalmic procedures on an eye, according to certain embodiments. FIG. 1B illustrate example subsystems of surgical console 100 of ophthalmic surgical system 10 of FIG. 1A, according to certain embodiments. FIGS. 1A–1B are described together herein for clarity.

In the illustrated embodiments, ophthalmic surgical system 10 includes surgical console 100 having a housing 102, a display screen 104, a wired or wireless interface device 107, a fluidics subsystem 110, cassette bay 118, and a hand piece 112. Housing 102 accommodates a computer 103 with an associated display screen 104 and one or more subsystems to support the interface device 107 and the hand piece 112. The interface device 107 receives input to the ophthalmic surgical system 10, sends output from the ophthalmic surgical system 10, and/or processes the input and/or output via the interface subsystem 106. In certain embodiments, the interface device 107 can include, without limitation, a foot pedal, manual input device (e.g., a keyboard), a display, and combinations of the same and like. In certain embodiments, a sensor system 120 may be integrated with the surgical console 100 via the interface subsystem 106. In certain embodiments, the sensor system 120 includes one or more sensors disposed in, or adjacent to, the cassette bay 118 that are configured to detect when a surgical cassette is attached to, and properly aligned within, the surgical console 100, and/or detect one or more fluid characteristics (such as pressure and volume) within the surgical cassette when attached to the surgical console 100. Detection of a properly aligned surgical cassette enables optimal cassette latching and engagement (e.g., fastening) with the fluidics module.

In certain embodiments, the hand piece 112 can include any suitable ophthalmic surgical instrument or device, such as, for example, an ultrasonically-driven phacoemulsification (phaco) hand piece, a laser hand piece, an irrigating cannula, a vitrectomy hand piece, or another suitable surgical hand piece. In various embodiments, the hand piece 112 can include a plurality of hand pieces 112, wherein at least two of the hand pieces 112 are different instruments or devices. The hand piece 112 can interact with the ophthalmic surgical system 10 via the hand piece subsystem 116. The fluidics subsystem 110 provides fluid control for the hand piece 112. For example, and not by way of limitation, the fluidics subsystem 110 can manage fluid for an irrigating cannula. In general, the surgical console 100 includes a hand piece subsystem that supports one or more hand pieces 112. For example, the hand piece subsystem can manage ultrasonic oscillation for a phaco hand piece, provide laser energy to a laser hand piece, control operation of an irrigating cannula, and/or manage features of a vitrectomy hand piece.

Computer 103 controls operation of ophthalmic surgical system 10. Generally, computer 103 includes a processor and a memory. The memory may include any device operable to receive, store, or recall data, including, but not limited to, electronic, magnetic, or optical memory, whether volatile or non-volatile. The memory may include code stored thereon. The code may include instructions that may be executable by the processor. The code may be created, for example, using any programming language, including but not limited to, C, C++, Java, Python, Rust, or any other programming language (including assembly languages, hardware description languages, and database programming languages). In some instances, the code may be a program that, when loaded into the processor, causes the surgical console 100 to receive and process information from one or more of subsystems 106, 110, and 116, for example, providing fluid control for one or more hand pieces 112 or other devices in communication with the surgical console 100.

The processor may be, or include, a microprocessor, a microcontroller, an embedded microcontroller, a programmable digital signal processor, or any other programmable device operable to receive information from the memory or other devices in communication with the processor, computer 103, and/or surgical console 100, and perform one or more operations on the received information. For example, the processor may send instructions to components of fluidics subsystem 110, or other devices or systems in communication with computer 103, for controlling such devices and systems. The processor may also be operable to output results based on the operations performed thereby. The display screen 104 shows data and other output results provided by the processor of computer 103. In some instances, the processor may also be or include an application specific integrated circuit, a programmable gate array, programmable array logic, or any other device of combinations of devices operable to process electric signals.

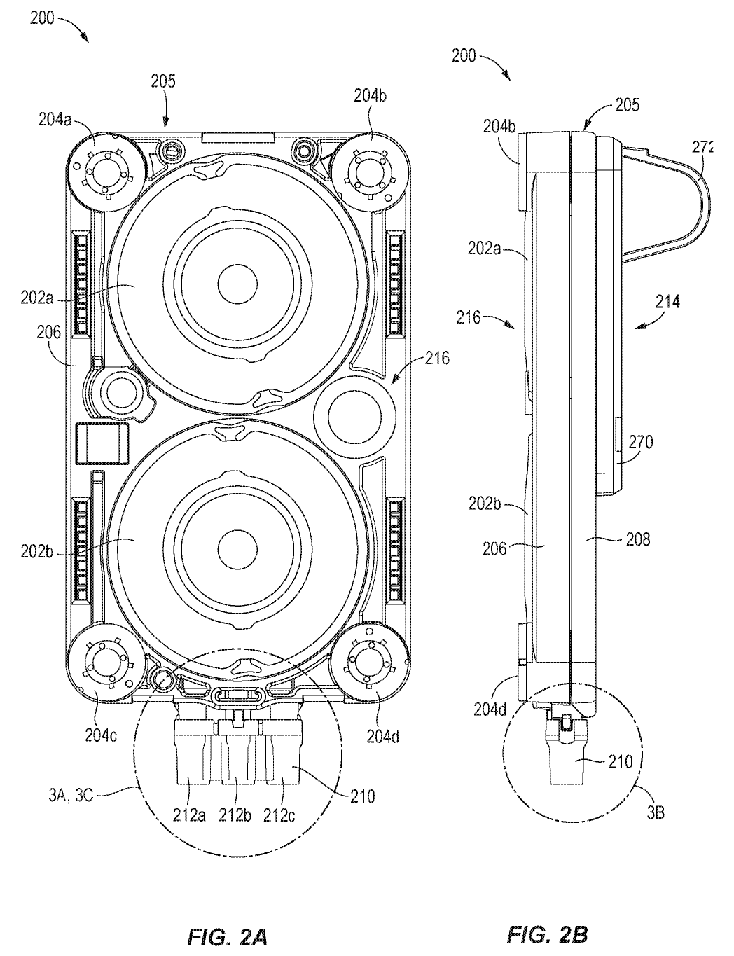

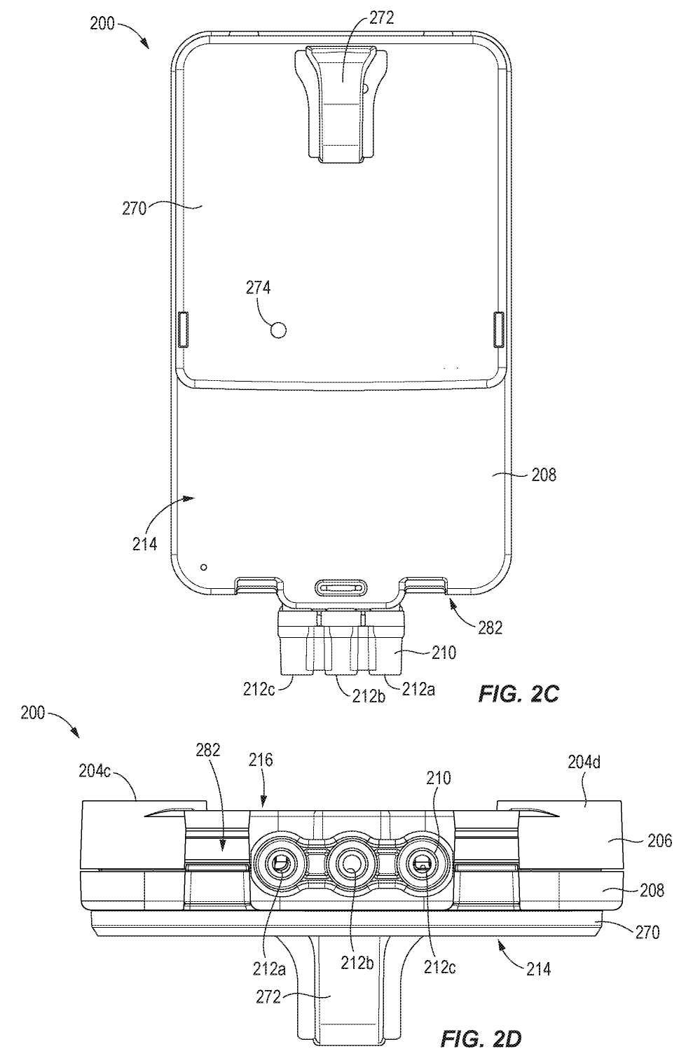

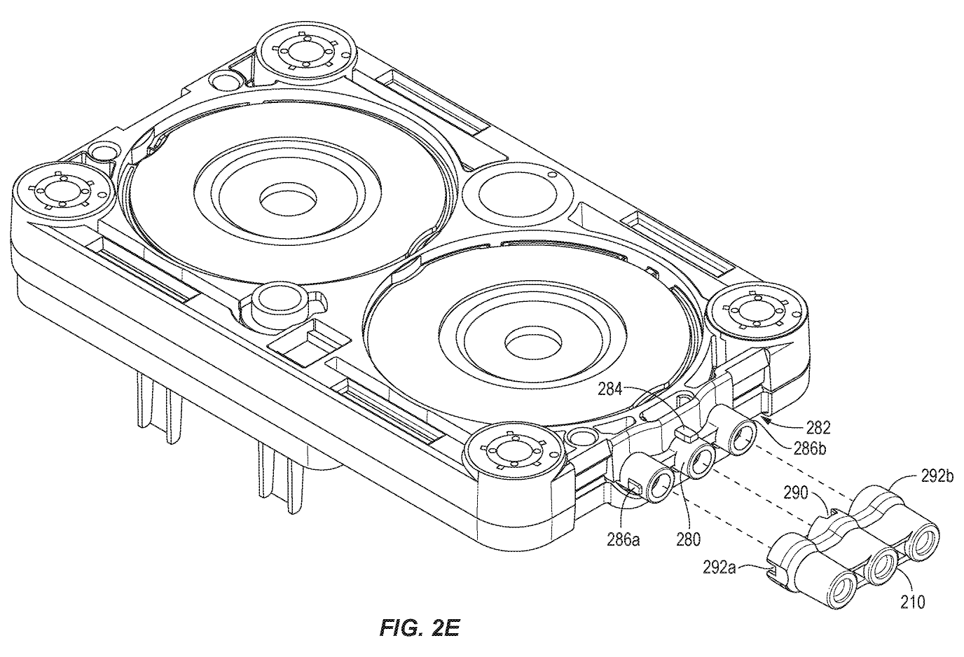

FIGS. 2A-2E show various views of an example surgical cassette 200 which may be operably coupled to a console of an ophthalmic surgical system (e.g., surgical console 100 of ophthalmic surgical system 10 illustrated in FIGS. 1A-1B), according to certain embodiments. FIG. 2A is a back plan view of the surgical cassette 200; FIG. 2B is a side elevation view of the surgical cassette 200; FIG. 2C is a front plan view of the surgical cassette 200; FIG. 2D is bottom-up elevation view of the surgical cassette 200; and FIG. 2E is a perspective exploded view of the surgical cassette 200. FIGS. 2A–2E are described together herein for clarity.

The surgical cassette 200 has a housing 205 including a base 206 and a cover 208 coupled to base 206. Generally, the base 206 is located on a back side 216 of the housing 205, which is visible in FIG. 2A. Meanwhile, the cover 208 is disposed on a front side 214 of the housing 205, which faces away from the back side 216 and is shown in FIG. 2C. In certain embodiments, the cover 208 is welded, bonded, or fastened to base 206 using any suitable coupling mechanism. For example, the cover 208 may be coupled to base 206 using a solid-state welding technique (e.g., ultrasonic welding in which high-frequency ultrasonic mechanical vibrations are locally applied to work parts being held together under pressure to create a solid-state weld).

In certain embodiments, an optional manifold 270 is attached to the cover 208. The manifold 270, also referred to as a “handle,” may serve, in part, to identify and/or determine a configuration or type of the surgical cassette 200. In certain embodiments, the manifold 270 includes a finger loop 272 to provide a physical handle or gripping surface for a user. In certain embodiments, the manifold 270 includes a drain port 274 to which a drain bag may be attached to receive and collect fluid waste, including aspirated materials, flowed through the surgical cassette 200, including the base 206, during a surgical procedure. In embodiments without the manifold 270, the drain port 274 may be formed in the cover 208.

In certain embodiments, the base 206, the cover 208, and/or the manifold 270 are formed of one or more of plastic, plastic composite, or thermoplastic materials. In certain embodiments, the base 206, the cover 208, and/or the manifold 270 are formed of the same material. In certain embodiments, the base 206, the cover 208, and/or the manifold 270 are formed of different materials.

In certain embodiments, one or more pump assemblies 202 are disposed in the base 206 for facilitating control of fluids through the surgical cassette 200 when the surgical cassette 200 is coupled to the surgical console 100. In the illustrated examples, two pump assemblies 202a and 202b are shown; however, other numbers of pump assemblies 202 are also contemplated. For example, in certain other embodiments, there may be only one pump assembly or more than two pump assemblies. Referring to FIGS. 2A-2E, in certain embodiments, one of the first pump assembly 202a or second pump assembly 202b provides a source of pressure (e.g., to create a driving force for fluid irrigation) while the other one of the first pump assembly 202a or second pump assembly 202b provides a source of vacuum (e.g., to create suction for fluid aspiration). The first pump assembly 202a and second pump assembly 202b may be peristaltic pumps or any other suitable type of pump for generating pressure and/or vacuum. In certain embodiments, the first pump assembly 202a and second pump assembly 202b are identical to each other.

In certain embodiments, an external source of pressure and/or vacuum is fluidly coupled to surgical cassette 200. In such embodiments, the external pressure and/or vacuum source may be either in place of, or in addition to, the pump assemblies 202.

One or more valve assemblies 204 are also disposed in the base 206. The valve assemblies 204 function cooperatively to control pressure and/or fluid communication within and through surgical cassette 200 in conjunction with the pump assemblies 202. In the illustrated embodiments, the surgical cassette 200 includes four valve assemblies 204: a first valve assembly 204a, a second valve assembly 204b, a third valve assembly 204c, and a fourth valve assembly 204d. However, other numbers of valve assemblies, such as less than four valve assemblies or more than four valve assemblies, are also contemplated. The four valve assemblies 204 in FIGS. 2A-2E are arranged at the four corners of housing 205 and surrounding the two pump assemblies 202 which are arranged towards a center of housing 205. However, in certain embodiments, pump assemblies 202 and valve assemblies 204 may have other suitable arrangements.

In certain embodiments, the valve assemblies 204 may be operated to selectively route fluid flow between multiple internal channels of housing 205. For example, first valve assembly 204a and third valve assembly 204c may be in pressure and/or fluid communication with first pump assembly 202a to provide aspiration (suction) during an operation, and second valve assembly 204b and fourth valve assembly 204d may be in pressure and/or fluid communication with second pump assembly 202b to provide irrigation (infusion) during the same operation. Generally, the surgical cassette 200 may include various internal channels, partitions, ports, surfaces, reservoirs, or fluid pockets for facilitating the fluid management functions noted above. Such channels may be formed in the base 206, the cover 207, and/or the manifold 270, and may be in fluid communication with one or more ports in the base 206, the cover 207, and/or the manifold 270.

In the illustrated embodiments, the back side 216 of the housing 205 is configured to interface with the surgical console 100 when the surgical cassette 200 is coupled thereto. For example, a drive interface on a valve body of each valve assembly 204 may engage a corresponding drive mechanism of the surgical console 100 for rotating the corresponding valve body. Meanwhile, the front side 214 of the housing 205 may be configured as a user interface for handling by a user.

In FIGS. 2A-2E, the surgical cassette 200 further includes a tubing adapter 210 that is welded (e.g., ultrasonically-welded), or otherwise coupled, to the base 206 and provides a connection point for one or more flexible fluidic tubes, and/or other types of fluid lines, to facilitate fluid flow between the surgical cassette 200 and corresponding component(s) of the fluidics subsystem 110 and/or corresponding hand piece(s) 112 (shown in FIGS. 1A-1B). This arrangement differs from conventional surgical cassettes, where the housings of such cassettes are typically bonded directly to fluidic tubes via bonding agents or other adhesives. However, the materials typically utilized for conventional cassette housings provide for weak adhesion with many types of bonding agents, which is further worsened with storage time and sterilization. Thus, with conventional surgical cassettes, there is an increased likelihood of fluidic tubes detaching from the cassettes during use in a surgical procedure.

In the present disclosure, the tubing adapter 210 functions as an improved interface that provides a more robust connection point between the housing 205 of the surgical cassette 200 and any fluidic tubing. In certain embodiments, to facilitate a strong connection between the surgical cassette 200 and corresponding fluidic tubes, the tubing adapter 210 is formed of a material that facilitates strong adhesion with bonding agents utilized to adhere the fluidic tubes to the tubing adapter 210, while further enabling welding of the tubing adapter 210 to the housing 205. For example, in certain embodiments, the tubing adapter 210 is formed of one or more thermoplastic polymers, such as polycarbonate, polypropylene, polyethylene, polyvinyl chloride, polystyrene, or the like. However, other types of materials for the tubing adapter 210 are also contemplated. Generally, the material of the tubing adapter 210 can be different from the material of the housing 205. For example, the housing 205 (e.g., the base 206) can be formed of a first thermoplastic material, and the tubing adapter 210 can be formed of a second thermoplastic material different than the first thermoplastic material. Yet, it is also contemplated that the material of the tubing adapter 210 can be the same as the material of the housing 205.

The tubing adapter 210 includes one or more ports 212, each port 212 configured to receive a fluidic tube for coupling with the surgical cassette 200. In the illustrated embodiments, the tubing adapter 210 includes three ports 212a, 212b, and 212c. In such embodiments, the port 212a may function as an aspiration port through which fluids are drawn into the surgical cassette 200 from a corresponding tube, the port 212c may function as an irrigation port through which fluids are drawn from the surgical cassette 200 into a corresponding tube, and the port 212b may function as an administration port configured to receive irrigation and/or infusion fluids to be delivered to the eye via the port 212c. During a surgical procedure, the first valve assembly 204a and the third valve assembly 204c may be in pressure and/or fluid communication with the first pump assembly 202a and the port 212a to provide aspiration (suction), and the second valve assembly 204b and the fourth valve assembly 204d may be in pressure and/or fluid communication with the second pump assembly 202b and the port 212c to provide irrigation (infusion) during the same operation.

Referring now to FIG. 2E, a perspective exploded view of the surgical cassette 200 is shown with the tubing adapter 210 separated from the base 206. As shown, the tubing adapter 210 couples to the base 206 of the surgical cassette 200 at a dock 280. In certain embodiments, such as in the examples of FIGS. 2A-2E, the dock 280 extends from a lateral surface 282 of the base 206. However, other arrangements for the dock 280 along the base 206 are also contemplated.

Generally, at least a portion of the tubing adapter 210 is configured to slide over the dock 280 such that the dock 280 at least partially nests within the tubing adapter 210 when coupled therewith. Accordingly, the dock 280 may be shaped and/or sized to be received within the tubing adapter 210, and at least a portion of the tubing adapter 210 may be shaped and/or sized to receive and slide over the dock 280.

In order to facilitate proper orientation of the tubing adapter 210 with the dock 280 during coupling (e.g., during fabrication of the surgical cassette 200), the dock 280 includes at least one medial key 284 configured to mate with a medial keyway 290 on the tubing adapter 210. The incorporation of the medial key 284 and medial keyway 290 ensures that the tubing adapter 210 is only coupled to the dock 280 in a single rotational orientation (e.g., only when the tubing adapter 210 “faces” a certain direction) during assembly. Accordingly, the medial key 284 and medial keyway 290 may be described as “orientation features” elsewhere herein.

In the illustrated embodiment, the medial key 284 includes a protrusion extending from a surface of the dock 280 that is disposed on, or adjacent to, the back side 216 of housing 205. Meanwhile, the medial keyway 290 includes a cutout formed through a portion of the tubing adapter 210 that is configured to be disposed on, or adjacent to, the back side 216 of housing 205. However, other types of features and arrangements for the medial key 284 and medial keyway 290 are also contemplated. For example, in certain embodiments, the medial key 284 and medial keyway 290 may be reversed such that the medial key 284 is disposed on the tubing adapter 210 and the medial keyway 290 is formed in the dock 280. In further embodiments, the medial key 284 and medial keyway 290 may be formed on surfaces of the dock 280 and tubing adapter 210 configured to be disposed on the front side 214 of the housing 205, in addition or alternatively to the back side 216.

In certain embodiments, the dock 280 includes at least one lateral key 286 configured to mate with a lateral keyway 292 on the tubing adapter 210. The lateral key 286 and lateral keyway 292 ensure proper alignment of the tubing adapter 210 with the dock 280 to allow for tighter-toleranced gaps therebetween, and to allow for adequate spacing between the tubing adapter 210 and the dock 280 at weld joints where the tubing adapter 210 is welded to the dock 280, as further described below. In other words, the incorporation of the lateral key 286 and lateral keyway 292 facilitates tighter control or tolerance between the tubing adapter 210 and the dock 280 during coupling, and provides proper spacing for welding material to flow between and bond the tubing adapter 210 and the dock 280. Accordingly, the lateral key 286 and lateral keyway 292 may be described as “alignment features” elsewhere herein.

In the illustrated embodiment, the dock 280 includes two lateral keys 286a and 286b (collectively referred to as lateral keys 286 elsewhere herein), and the tubing adapter 210 includes corresponding lateral keyways 292a and 292b (collectively referred to as lateral keyways 292 elsewhere herein), respectively. The lateral keys 286 include protrusions extending from surfaces disposed between the back side 216 and the front side 214 of the housing 205 and at opposing ends of the dock 280. Similarly, the lateral keyways 292 include cutouts formed at opposing ends of the tubing adapter 210 that are configured to be disposed between the back side 216 and the front side 214. However, other types of features and arrangements for the lateral keys 286 and lateral keyways 292 are further contemplated. For example, in certain embodiments, the lateral keys 286 and lateral keyways 292 may be reversed such that the lateral keys 286 are disposed on the tubing adapter 210 and the lateral keyways 292 are formed in the dock 280. Generally, having two lateral keys 286 on opposing ends of the dock 280 and two lateral keyways 292 on opposing ends of the tubing adapter 210 ensures that the same tolerances or gaps are formed between both ends of the dock 280 and the corresponding ends of the tubing adapter 210.

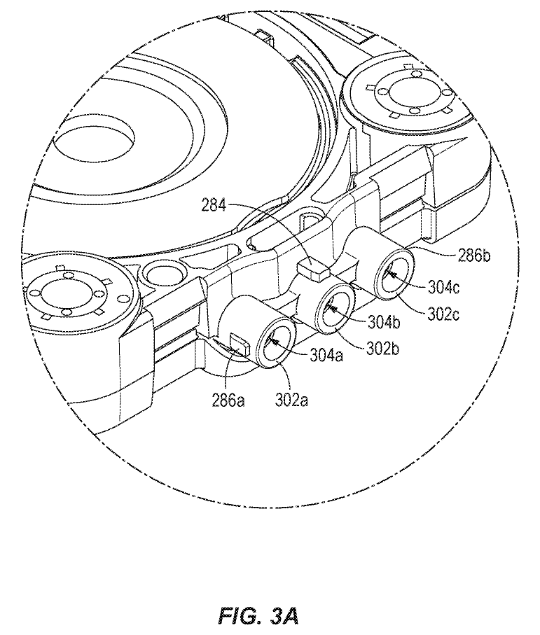

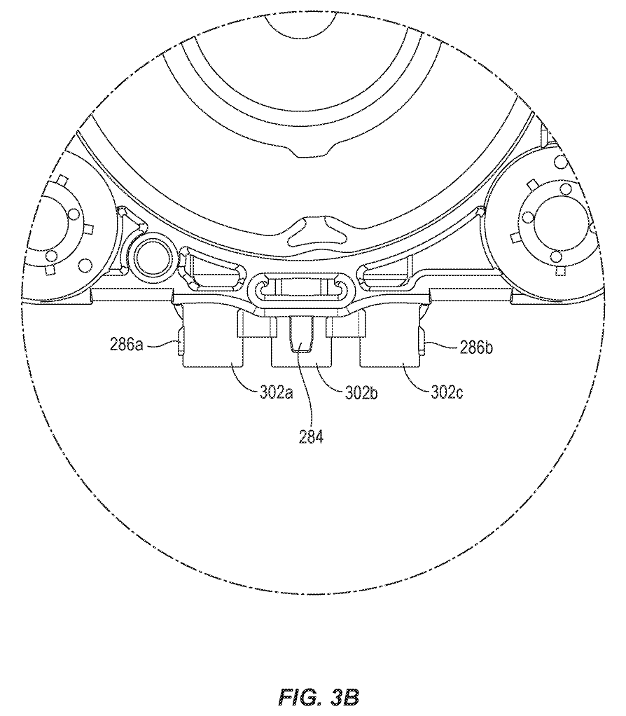

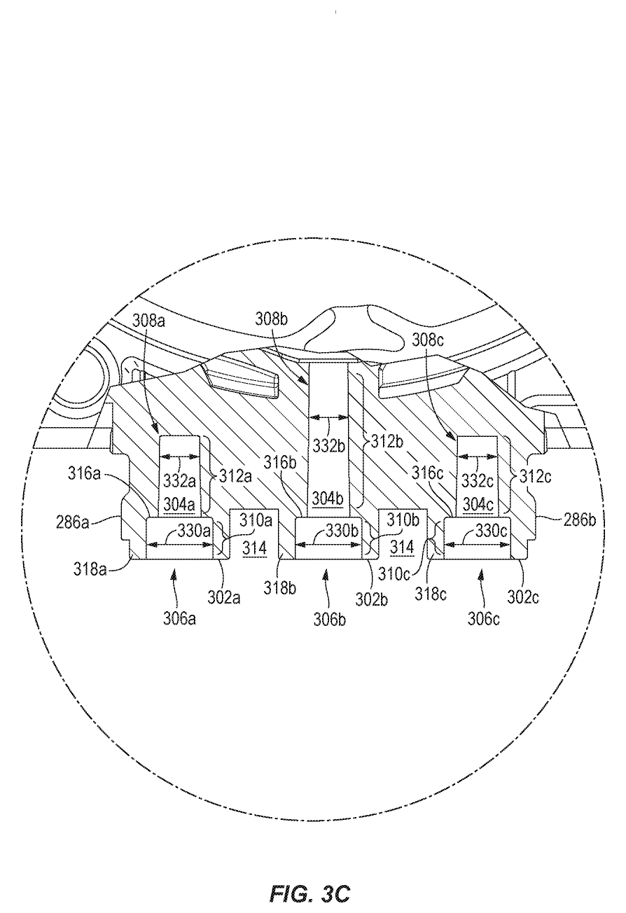

FIGS. 3A-3C are various views of the dock 280 of the surgical cassette 200, according to certain embodiments. FIG. 3A is an enlarged perspective view of the dock 280; FIG. 3B is a back plan view of the dock 280; and FIG. 3C is a cross-sectional view of dock 280. FIGS. 3A–3C are described together herein for clarity.

As shown, the dock 280 includes three connectors 302a, 302b, and 302c (collectively referred to as connectors 302 elsewhere herein) disposed in a linear arrangement. Each of the connectors 302 is configured to engage with, or mate with, a corresponding port 212 of the tubing adapter 210 during assembly of the surgical cassette 200, as described in further detail below.

Generally, each connector 302 includes a channel 304 extending through a wall 318 (walls 318a, 318b, and 318c and channels 304a, 304b, and 304c are illustrated, corresponding with each of the connectors 302a, 302b, and 302c, respectively), which may generally be annular or tubular in shape. The channels 304 extend between distal ends 306 and proximal ends 308 of the connectors 302 (distal ends 306a, 306b, and 306c and proximal ends 308a, 308b, and 308c are illustrated, corresponding with each of the connectors 302a, 302b, and 302c, respectively). Generally, the proximal ends 308 lead into, or are fluidly coupled with, an interior of the housing 205 of the surgical cassette 200, which includes one or more channels, partitions, ports, surfaces, reservoirs, or fluid pockets. Accordingly, the channels 304 facilitate pressure and/or fluid communication between an interior and exterior of the housing 205 during use of the surgical cassette 200.

In certain embodiments, the channels 304 are variable in lateral dimensions (e.g., diameter or width) between the distal ends 306 and proximal ends 308. For example, each channel 304 can include a distal segment 310 and a proximal segment 312, wherein the distal segment 310 has a first lateral dimension 330 greater than a second lateral dimension 332 of the proximal segment 312 (distal segments 310a, 310b, and 310c, proximal segments 312a, 312b, and 312c, first lateral dimensions 330a, 330b, and 330c, and second lateral dimensions 332a, 332b, and 332c are illustrated, corresponding with each of the channels 304a, 304b, and 304c, respectively). In such embodiments, the distal segments 310 may be configured to receive, or mate with, a portion of the tubing adapter 210, while the proximal segments 312 may be configured to function as a connecting fluid pathway between the tubing adapter 210 and the interior of the housing 205 during use of the surgical cassette 200. In certain embodiments, a ledge 316 is formed between each distal segment 310 and each proximal segment 312 of the channels 304 as a result of the varying dimensions, against which the tubing adapter 210 may contact and/or weld to during assembly. Generally, to form the variable dimensions of the channels 304, the thicknesses of the tubular walls 318 of the connectors 302 defining the channels 304 can be varied.

Also shown in FIGS. 3A-3C are medial key 284 and lateral keys 286a and 286b. In the illustrated embodiments, the medial key 284 extends from an external surface 320b of the connector 302a on a side corresponding with the back side 216 of the housing 205. Meanwhile, the lateral keys 286a and 286b extend from external surfaces 320a and 320c of the connectors 302a and 302c, respectively, and between the back side 216 and the front side 214 of the housing 205. Further, the walls 318a and 318b, and the walls 318b and 318c, are shown as being separated from each other via spaces 314 at distal ends 306 of the corresponding connectors 302.

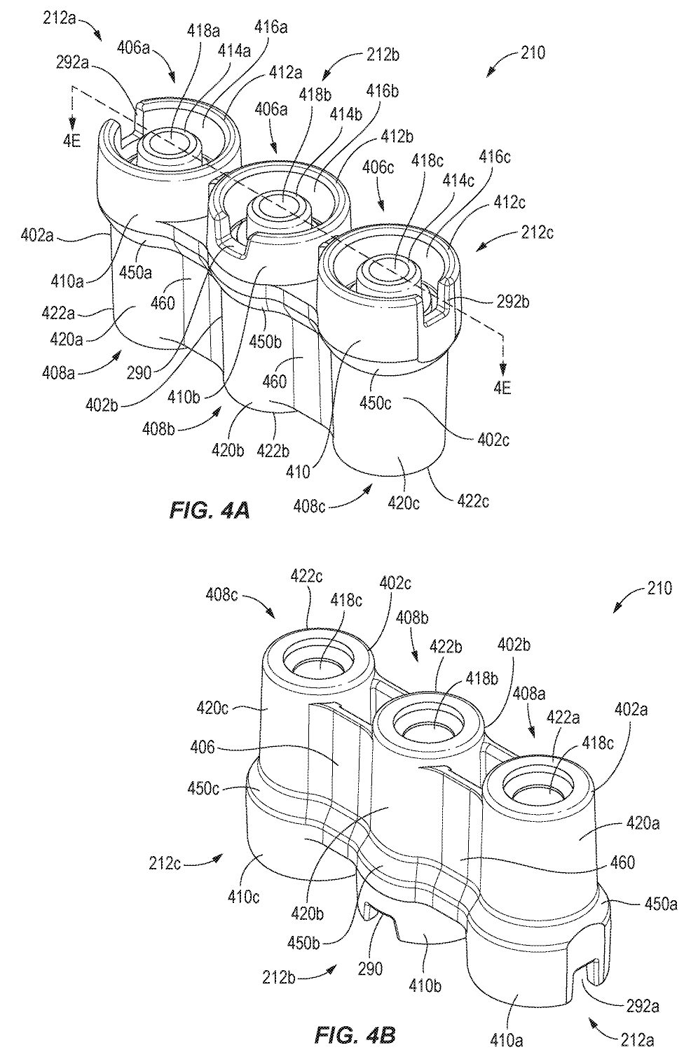

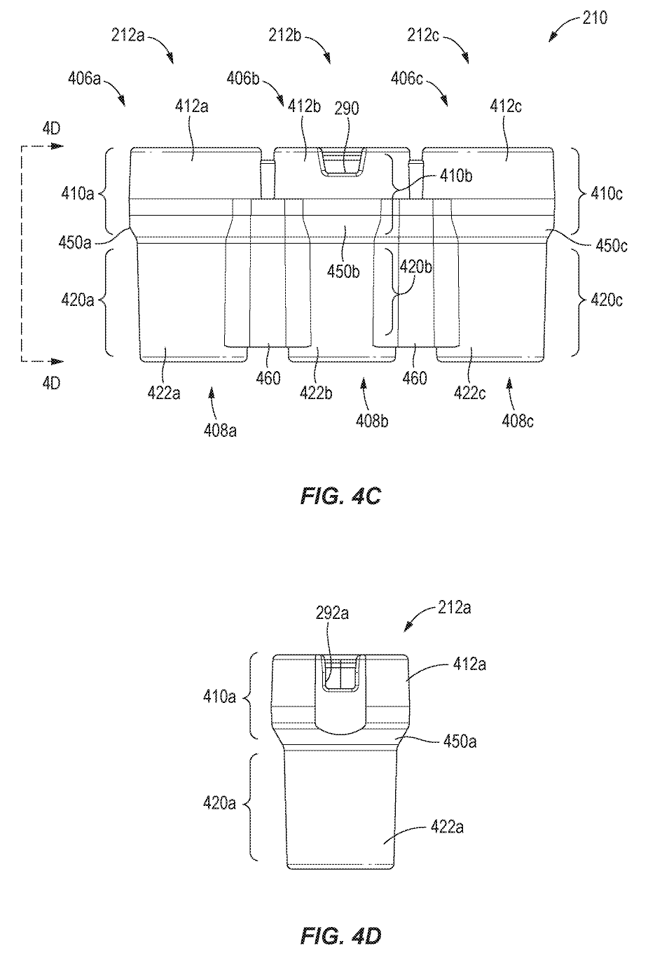

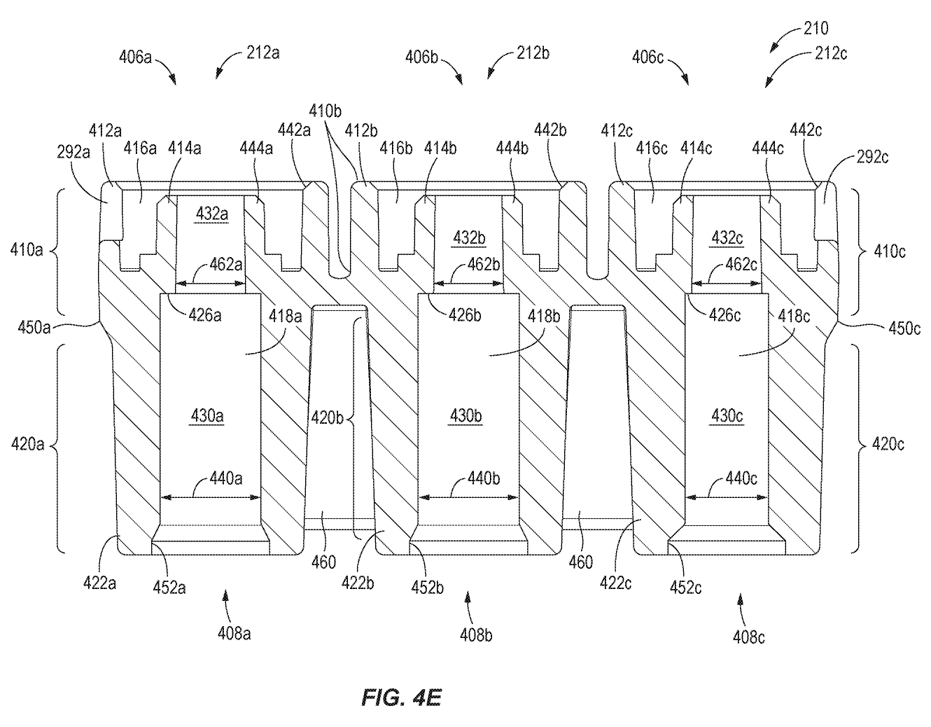

FIGS. 4A-4D are various views of the tubing adapter 210 of the surgical cassette 200, according to certain embodiments. FIG. 4A is an enlarged perspective view of the tubing adapter 210; FIG. 4B is another enlarged perspective view of the tubing adapter 210; FIG. 4C is a back plan view of the tubing adapter 210; FIG. 4D is an elevated view of the tubing adapter 210; and ; FIG. 4E is a cross-sectional view of the tubing adapter 210. FIGS. 4A–4D are described together herein for clarity.

As shown, the tubing adapter 210 includes the three ports 212a, 212b, and 212c (collectively referred to as ports 212 elsewhere herein) coupled to each other by two or more bridges 460. Each of the ports 212 is configured to engage with, or mate with, a corresponding connector 302 of the dock 280 during assembly of the surgical cassette 200, as described in further detail below. For example, the port 212a is configured to mate with the connector 302a; the port 212b is configured to mate with the connector 302b; and the port 212c is configured to mate with the connector 302c.

Each port 212 includes a proximal end 406 and an opposing distal end 408, and a channel 418 extending therebetween (proximal ends 406a, 406b, and 406c, distal ends 408a, 408b, and 408c, and channels 418a, 418b, and 418c are illustrated, corresponding with each of the ports 212a, 212b, and 212c, respectively).

As best seen in FIGS. 4A and 4E, each of the ports 212 further includes a first portion 410 at the proximal end 406 configured to interface with the connectors 302 of the dock 280 (first portions 410a, 410b, and 410c are illustrated, corresponding with each of the ports 212a, 212b, and 212c, respectively). The first portion 410 includes an outer wall 412 and an inner wall 414 separated by a gap 416 (outer walls 412a, 412b, and 412c, inner walls 414a, 414b, and 414c, and gaps 416a, 416b, and 416c are illustrated, corresponding with each of the first portions 410a, 410b, and 410c, respectively). One or more surfaces 424 extends between the outer wall 412 and the inner wall 414 at a distal end of the gap 416 in each port 212 (surfaces 424a, 424b, and 424c are illustrated, corresponding with each of the first portions 410a, 410b, and 410c, respectively). In the illustrated embodiments, each of the outer wall 412, inner wall 414, gap 416, and surface(s) 424 are annular in shape; however, other shapes are also contemplated.

During assembly, the walls 318 of the connectors 302 are configured to be received within the gaps 416 of the ports 212 such that each of the outer walls 412 slides over a corresponding wall 318 and each of the inner walls 414 slides into a corresponding channel 304. Accordingly, the walls 318 of the connectors 302 may be shaped and/or sized to be received within the gaps 416 of the ports 212, and the inner walls 414 of the ports 212 may be shaped and/or sized to be received within the channels 304 of the connectors 302. In certain embodiments, proximal ends of the outer walls 412 and inner walls 414 (e.g., ends of the outer walls 412 and inner walls 414 nearest the proximal ends 406) are rounded, tapered, or sloped to facilitate easier alignment and attachment of the ports 212 to corresponding connectors 302. For example, as shown in FIG. 4E, outer walls 412 include tapered medial surfaces 442 and inner walls 414 include tapered outer surfaces 444 to facilitate the alignment and engagement thereof with the walls 318 of the connectors 302 (medial surfaces 442a, 442b, and 442c and outer surfaces 444a, 444b, and 444c are illustrated, corresponding with each of the first portions 410a, 410b, and 410c, respectively).

As best seen in FIGS. 4B and 4E, each of the ports 212 further includes a second portion 420 at the proximal end 406 configured to interface with flexible tubes, and/or other types of fluid lines (second portions 420a, 420b, and 420c are illustrated, corresponding with each of the ports 212a, 212b, and 212c, respectively). Each second portion 420 is formed of a singular wall 422 from which the inner wall 414 and the outer wall 412 of the corresponding first portion 410 extend proximally (walls 422a, 422b, and 422c are illustrated, corresponding with each of the second portions 420a, 420b, and 420c, respectively). Similar to the inner walls 414 and the outer walls 412, the walls 422 can have a generally annular, or tubular, shape. Together with the inner walls 414, the walls 422 define the channels 418 extending through the ports 212.

In certain embodiments, the outer dimensions of the first portions 410 and corresponding second portions 420 are the same. As shown in the illustrated embodiments, in certain embodiments, the outer dimensions of the first portions 410 and corresponding second portions 420 are different. For example, in such embodiments, the first portions 410 may have larger lateral dimensions (e.g., diameters) than the corresponding second portions 420, or vice versa. In such embodiments, the ports 212 include transition portions 450 disposed between the first portions 410 and the second portions 420 (transition portions 450a, 450b, and 450c are illustrated, corresponding with each of the ports 212a, 212b, and 212c, respectively). Each transition portion 450 may generally include a tapered outer surface extending between the outer wall 412 of a first portion 410 and the wall 422 of a corresponding second portion 420.

When assembled (e.g., the first portions 410 are attached to corresponding connectors 302, and the second portions 420 are coupled to fluidic tubing), the channels 418 provide fluidic pathways for fluid flow between the surgical cassette 200 and corresponding components of the fluidics subsystem 110 and/or hand pieces 112 of the surgical system 10. Referring to FIG. 4E, in certain embodiments, the channels 418 are variable in lateral dimensions (e.g., diameter or width) between the proximal ends 406 and distal ends 408. For example, in FIG. 4E, each channel 418 has a distal segment 430 through the second portion 420 and a proximal segment 432 through the first portion 410, wherein the distal segment 430 has a first lateral dimension 440 greater than a second lateral dimension 462 of the proximal segment 432. In such embodiments, the distal segments 430 are configured to receive the proximal ends of fluidic tubes during assembly, while the proximal segments 432 are configured to function as a connecting fluid pathway between the tubes and corresponding channels 304 of the connectors 302. In certain embodiments, the lateral dimensions 440 of the distal segments 430 of the channels 418 are sized to substantially match the outer dimensions of the proximal ends of the fluidic tubes to be received within the channels 418. Meanwhile, the lateral dimensions 462 of the proximal segments 432 are sized to substantially match the inner dimensions of the fluidic tubes disposed through the distal segments 430 in order to form a streamlined fluid path between the fluidic tubes and the interior of the surgical cassette 200, without creating any blockage of fluids. In certain embodiments, a ledge 426 is formed between each distal segment 430 and the corresponding proximal segment 432 of the channels 418 as a result of the varying dimensions, against which a proximal end of a fluidic tube may contact during assembly.

In certain embodiments, distal ends of the second segments 430 (e.g., ends of the segments 430 nearest the distal ends 408) are tapered to facilitate easier alignment and insertion of fluidic tubes into the channels 418. For example, as shown in FIG. 4E, in certain embodiments, each of the second segments 430 includes a tapered portion 452 wherein the channel 418 reduces in lateral dimensions proximally (tapered portions 452a, 452b, and 452c are illustrated, corresponding with each of the channels 418a, 418b, and 418c, respectively).

In certain embodiments, the tubing adapter 210 is monolithic in structure. In such embodiments, the tubing adapter 210 is fabricated via injection molding, three-dimensional (3D) printing, or similar techniques. In certain other embodiments, however, the tubing adapter 210 is polylithic, and one or more components thereof are fabricated individually and then attached together (e.g., via an adhesive or other bonding agent).

The first portion 410 includes an outer wall 412 and an inner wall 414 separated by a gap 416 (outer walls 412a, 412b, and 412c, inner walls 414a, 414b, and 414c, and gaps 416a, 416b, and 416c are illustrated, corresponding with each of first portions 410a, 410b, and 410c, respectively). In the illustrated embodiments, each of the outer wall 412, inner wall 414, and gap 416 are annular in shape; however, other shapes are also contemplated. During assembly, the walls 318 of the connectors 302 are configured to be received within the gaps 416 of the ports 212 such that each of the outer walls 412 slides over a corresponding wall 318 and each of the inner walls 414 slides into a corresponding channel 304. Accordingly, the walls 318 of the connectors 302 may be shaped and/or sized to be received within the gaps 416 of the ports 212, and the inner walls 414 of the ports 212 may be shaped and/or sized to be received within the channels 304 of the connectors 302.

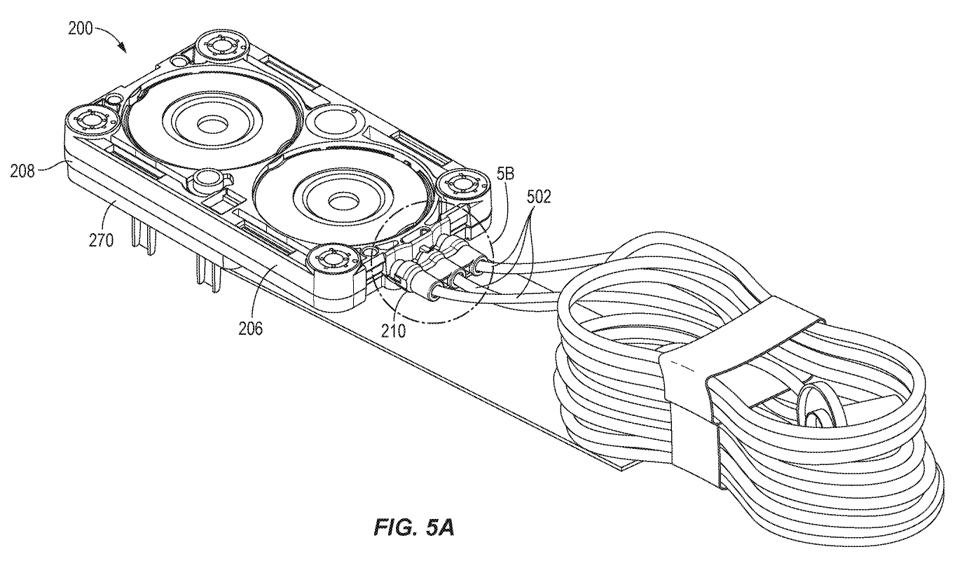

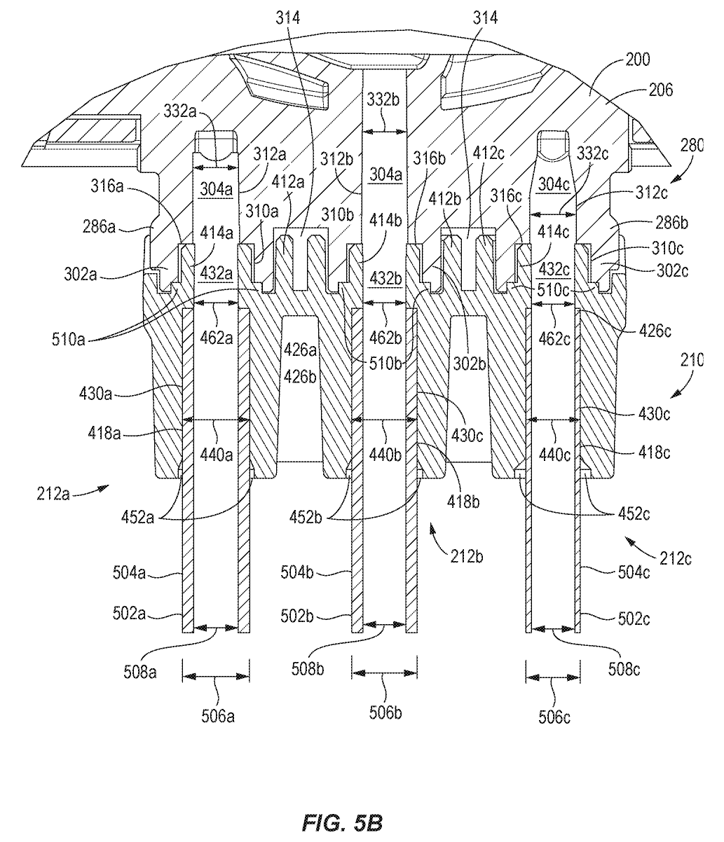

FIGS. 5A and 5B are various views of the surgical cassette 200, including the tubing adapter 210, attached to fluidic tubes 500, according to certain embodiments. FIG. 5A is a perspective view of the surgical cassette 200 and the fluidic tubes 500; FIG. 5B is an enlarged cross-sectional view of a portion of the surgical cassette 200, including the tubing adapter 210 and the fluidic tubes 500. FIGS. 5A–5B are described together herein for clarity.

When assembled, the first portions 410 of the ports 212 of the tubing adapter 210 are mated with the distal ends 306 of the connectors 302 of the dock 280. The outer walls 412 of the ports 212 slide over the corresponding walls 318 of the connectors 302, while the inner walls 414 of the ports 212 slide into the distal segments 310 of the corresponding channels 304 of the connectors 302. As a result, the walls 318 of the connectors 302 are received within the gaps 416 of the ports 212, while the outer walls 412 of the ports 212 are received in the spaces 314 between adjacent connectors 302. In certain embodiments, when fully engaged, proximal ends of the inner walls 414 of the ports 212 are disposed adjacent to, or come into contact with, the ledges 316 of the connectors 302. Similarly, distal ends of the walls 318 are disposed adjacent to, or come into contact with, the surfaces 424 of the ports 212.

To fixedly attach the tubing adapter 210 to the dock 280, the tubing adapter 210 can be welded to the dock 280 after mating the ports 212 with the corresponding connectors 302. In certain embodiments, the tubing adapter 210 can be welded to the dock 280 via ultrasonic welding, in which high-frequency ultrasonic mechanical vibrations are locally applied to work parts being held together under pressure to create a solid-state weld. For example, the tubing adapter 210 can include one or more weld joints 510, such as steps, protrusions, grooves, and the like, that are configured to “melt” upon application of ultrasonic vibration and bond with adjacent surfaces of the dock 280. In the illustrated embodiments, the weld joints 510 include annular ledges of, or annular ledges disposed adjacent to, the surfaces 424 between the outer walls 412 and inner walls 414 of the ports 212 (three weld joints 510a, 510b, and 510c are shown, corresponding to each port 212a, 212b, and 212c, respectively). During welding, the material of the weld joints 510 can be “melted” to travel through gaps between the tubing adapter 210 and the dock 280 and bond the two components together. As noted above, for optimal bonding of the tubing adapter 210 to the dock 280 during welding, the gaps created therebetween can be tightly toleranced during assembly, which is facilitated by the alignment and orientation features of the tubing adapter 210 and the dock 280. In certain embodiments, it is further contemplated that the dock 280 can include weld joints 510 in addition to, or alternatively to, the tubing adapter 210.

The tubing adapter 210 is also attached to one or more fluidic tubes 502 during fabrication and prior to utilization in a surgical procedure. The fluidic tubes 502 can include any suitable type of fluidic tubing. For example, in certain embodiments, the fluidic tubes 502 include surgical grade, flexible fluidic tubing, such tubing formed from non-diethylhexyl phthalate (DEHP) polyvinyl chloride (PVC), and/or other types of nontoxic and/or phthalate-free thermoplastic polymers.

In the illustrated embodiments, three fluidic tubes 502a, 502b, and 502c are depicted (collectively referred to as fluidic tubes 502 elsewhere herein), each attached to a corresponding port 212a, 212b, and 212c of the tubing adapter 210, respectively. As noted above, the port 212a may function as an aspiration port, the port 212c may function as an irrigation port, and the port 212b may function as an administration port. Accordingly, in such embodiments, the fluidic tube 502a may function as a conduit through which fluids are drawn into the surgical cassette 200 from a component of the fluidics subsystem 110 or hand piece 112, the fluidic tube 502c may function as a conduit through which fluids are drawn from the surgical cassette 200 and into a component of the fluidics subsystem 110 or hand piece 112, and the fluidic tube 502b may function as a conduit through which irrigation and/or infusion fluids are passed to the surgical cassette 200.

When assembled, a proximal end 504 of each fluidic tube 502 is received in the distal segment 430 of the channel 418 of a corresponding port 212 (three proximal ends 504a, 504b, and 504c are shown, corresponding to each fluidic tube 502a, 502b, and 502c, respectively). In certain embodiments, the fluidic tubes 502 can be inserted into the distal segments 430 such that the proximal ends 504 thereof are disposed adjacent to, or contact against, the ledges 426 between the distal segments 430 and proximal segments 432 of the channels 418. To fixedly attach the fluidic tubes 502 within the channels 418, a bonding agent may be used. For example, a bonding agent may be applied to external surfaces of the fluidic tubes 502 prior to inserting of the fluidic tubes 502 into the distal segments 430 of the channels 418. The bonding agent can then adhere the fluidic tubes 502 to the inner surfaces of the distal segments 430 once the fluidic tubes 502 are inserted therein. In certain embodiments, the bonding agent includes at least one of acetone, cyanoacrylate, cyclohexanone, dichloromethane, dimethylformamide, ethyl acetate, ethylene dichloride, methyl isobutyl ketone, methylethylketone, tetrahydrofuran, toluene, xylene, or other solvent. In certain embodiments, upon insertion of the fluidic tubes 502 into the channels 418, the bonding agent may accumulate within gaps disposed between surfaces of the tapered portions 452 of the channels 418 and external surfaces of the fluidic tubing 502. Accordingly, the tapered portions 452 may function to facilitate pooling of the bonding agent outside of the fluidic pathway between the fluidic tubes 502, tubing adapter 210, and dock 280.

As shown in FIG. 5B, the lateral dimensions 440 of the distal segments 430 of the channels 418 are sized to substantially match, and/or be slightly larger than, the outer dimensions 506 of the fluidic tubes 502. Meanwhile, the lateral dimensions 462 of the proximal segments 432 of the channels 418 are sized to substantially match the inner dimensions 508 of the fluidic tubes 502, as well as the lateral dimensions 332 of the channels 304 of the dock 280. As a result of this arrangement, a streamlined fluid path is formed between each of the fluidic tubes 502 and the interior of the surgical cassette 200, without creating any blockage or turbulence of fluids passing therethrough. In some examples, the uniform (e.g., unchanging) cross-sections of the interior lumens of the fluidic tubes 502 and the corresponding channels 418 facilitate stable fluid flow paths between the fluidic tubes 502 and the interior of the surgical cassette 200 with consistent fluid flow speed and pressure, while preventing or at least reducing pressure loss, eddy currents, and the trapping of bubbles.

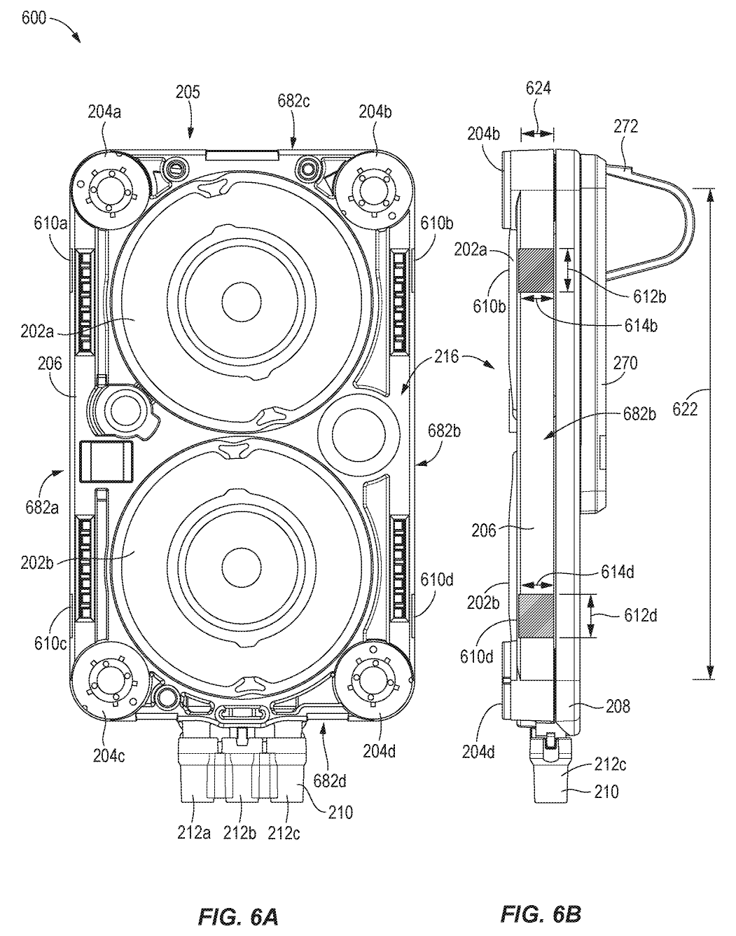

FIGS. 6A and 6B are various views of an example surgical cassette 600 which may be operably coupled to the surgical console 100 of the surgical system 10, according to certain embodiments. FIG. 6A is a back plan view of the surgical cassette 600; and FIG. 6B is an elevation view of the surgical cassette 600. FIGS. 6A–6B are described together herein for clarity.

The surgical cassette 600 is similar to the surgical cassette 200. Accordingly, similar features are labeled with like reference numerals and are not described below for concision.

In addition to the features described above with reference to the surgical cassette 200, the surgical cassette 600 may include a plurality of sensing regions 610. The sensing regions 610 facilitate accurate and consistent detection of the surgical cassette 600 when attached to the surgical console 100, and mitigate any potential detection issues caused by manufacturing and/or material variability in the components of the surgical cassette 600. Where conventional surgical cassettes may be unreliably detected by surgical console sensors upon attachment due to variability in surface finish or composition of components as caused by fabrication processes, the sensing regions 610 provide consistent, invariable, and dependable features for detection by sensors of the surgical console 100. The sensing regions 610 further enable detection of proper alignment of the surgical cassette 600 when disposed within the cassette bay 118 of the surgical console 100.

Turning to FIGS. 6A and 6B, the sensing regions 610 are disposed on one or more of four lateral surfaces 682a, 682b, 682c, and 682d (collectively referred to as lateral surfaces 682 elsewhere herein) of the base 206 of the surgical cassette 600. In the illustrated embodiment of FIG. 6A, the surgical cassette 600 includes four sensing regions 610a-610d (collectively referred to as sensing regions 610 elsewhere herein), with two sensing regions 610 on each of the lateral surfaces 682a and 682b. However, other numbers and arrangements for the sensing regions 610 along the lateral surfaces 682a-682d are also contemplated, depending on the number and arrangement of sensors in the corresponding cassette bay 118 of the surgical console 100. For example, in certain embodiments, the surgical cassette 600 can include a single sensing region 610 on each of lateral surfaces 682a and 682b. In certain other embodiments, the surgical cassette 600 can include a single sensing region 610 on a single lateral surface 682. In further embodiments, one or more sensing regions 610 are disposed on the cover 208 or manifold 270 of the surgical cassette 600.

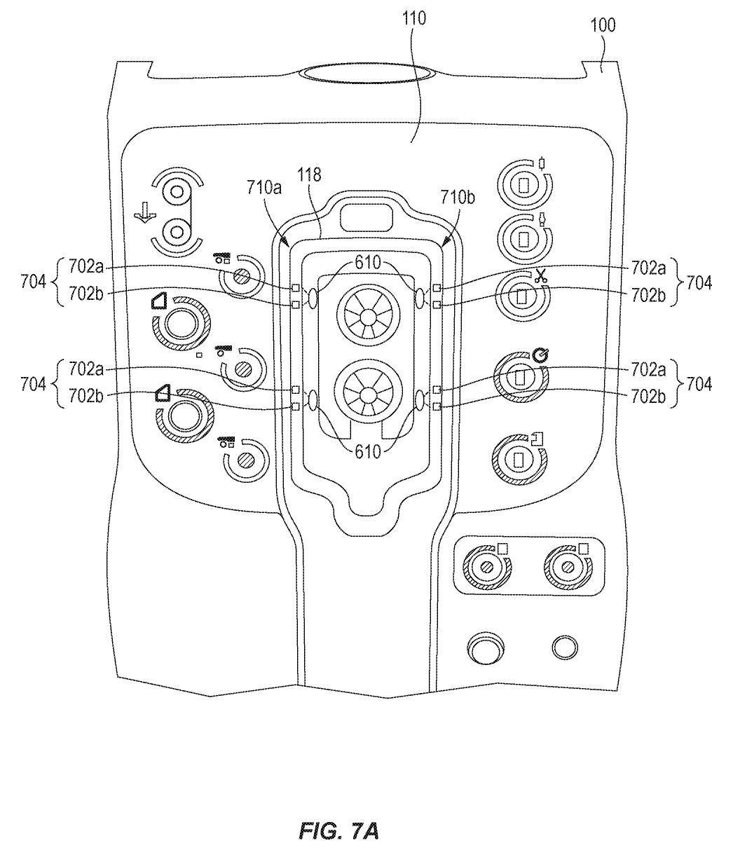

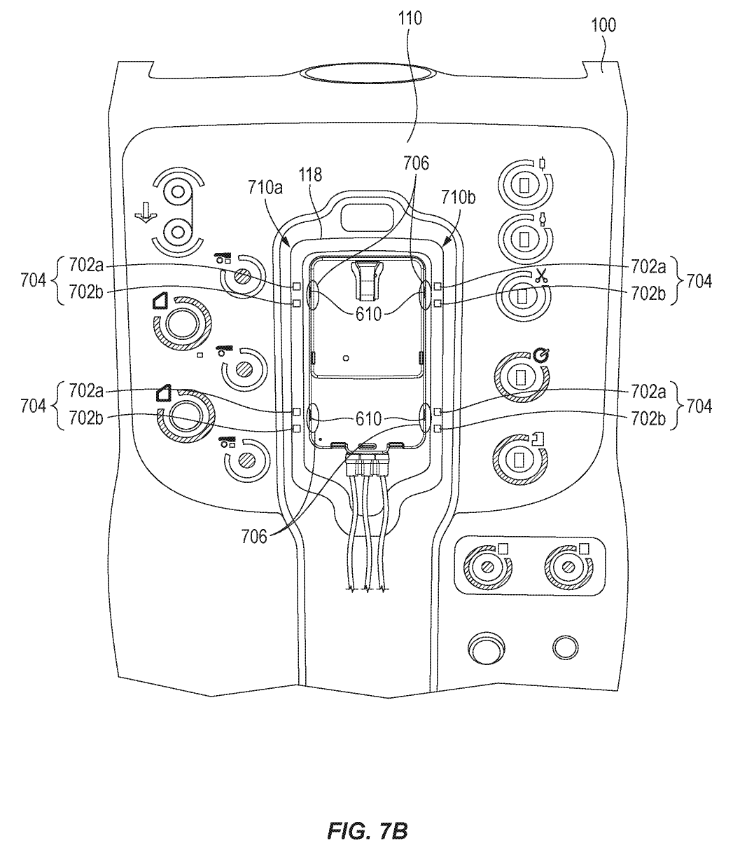

Generally, each of the sensing regions 610 includes a surface area of the surgical cassette 600 that can be detected by a sensor of the surgical console 100 to determine when the surgical cassette 600 is attached to, or loaded onto, the cassette bay 118, as further described with reference to FIGS. 7A and 7B. In certain embodiments, the sensing regions 610 include at least one of a material, surface finish, or coating that is non-transmissive to, and/or reflective of, target wavelengths configured to be detected by one or more optical sensors of the surgical console 100. In such embodiments, the material, surface finish, or coating can reduce or prevent the transmission of target wavelengths of light through the surgical cassette 600, and/or reduce or prevent the scattering of the target wavelengths, and/or reflect or increase the reflection of the target wavelengths to a corresponding optical sensor of the surgical console 100 for detection. In certain embodiments, the material, surface finish, or coating provides the sensing regions 610 with different light propagation/scattering properties as compared to other areas of the surfaces 682.

In certain embodiments, the target wavelengths of light include wavelengths of infrared (IR) light, such as wavelengths between 780 nm (nanometers) and 1 mm (millimeters). In certain embodiments, the target wavelengths of light include wavelengths of ultraviolet (UV) light, such as wavelengths between 100 nm and 400 nm. In certain embodiments, the target wavelengths include wavelengths of visible light, such as wavelengths between 400 and 700 nm. Generally, the target wavelengths of light may depend on the type of sensors utilized on the surgical console 100 to detect attached sensing regions 610 of the surgical cassette 600.

In certain embodiments, the surface finish of the sensing regions 610 includes a polished surface finish configured to reduce scattering and transmission of target wavelengths of light. Generally, the polished surface finish can be formed via any suitable polishing techniques, including mod steel polishing techniques.

In certain embodiments, the sensing regions 610 include polished surface finishes with a Society of the Plastic Industry (SPI) surface finish grade of SPI A1, SPI A2, or SPI A3. In such embodiments, the surface finish roughness of the sensing regions 610 can be between about 0.012 Ra (in micrometers (µm)) to about 0.10 Ra, or between about 0.012 Ra to about 0.025 Ra, or between about 0.012 Ra to about 0.10 Ra. Meanwhile, other outer surfaces of the surgical cassette 600 (surfaces other than the sensing regions 610), can have a surface roughness of about 20 Ra or more, 25 Ra or more, or 30 Ra or more, such as 31 Ra.

In certain embodiments, one or more of the sensing regions 610 may, in addition to or alternatively to polished surface finishes, include one or more of: a metallic paint applied thereto; a chrome, nickel, or other metallic layer plated thereon; an anodized layer formed thereon; a pad printed thereon with reflective ink; a reflective or metallic foil hot stamped thereon; an in-mold foil decoration (IMD) formed therein; or a reflective adhesive label applied thereto.

With reference to FIG. 6B, the sensing regions 610b and 610d on the lateral surface 682b of the base 206 of surgical cassette 600 are shown. Note that the description of the sensing regions 610b and 610d and the lateral surface 682b above can also apply to the sensing regions 610a and 610c and lateral surface 682a. The sensing regions 610 can have any suitable shape(s) and/or dimensions. For example, the sensing regions 610 can be rectangular, square, polygonal, nonpolygonal, circular, ovoid, regular, or irregular in shape. In the illustrated embodiments, each of the sensing regions 610b and 610d has a rectangular shape with a length 612 and a width 614 (lengths 612b and 612d and widths 614b and 614d, corresponding to sensing regions 610b and 610d, respectively, are shown), while the lateral surface 682b has a total length 622 and a total width 624. In certain embodiments, each of the lengths 612 and/or each of the widths 614 is less than the total length 622 and/or the total width 624 of the lateral surface 682b, respectively. In certain embodiments, each of the widths 614 is equal to, or approximately equal to, the total width 624. Where the lateral surface 682b includes a single sensing region 610b or 610d, the length 612b or 612d may be equal to, or approximately equal to, the total length 612.

In certain embodiments, the lengths 612 and widths 614 of the sensing regions 610 can be based on the dimensions of target sensing areas of corresponding sensors on the surgical console 100. For example, in certain embodiments, the lengths 612 and widths 614 of the sensing regions 610 are larger than the dimensions of the target sensing areas of the corresponding sensors on the surgical console 100. In certain embodiments, the lengths 612 and widths 614 of the sensing regions 610 correspond to the dimensions of the target sensing areas of the corresponding sensors on the surgical console 100.

FIGS. 7A and 7B are various views of the cassette bay 118 of the surgical console 100, without and with the surgical cassette 600 attached thereto, according to certain embodiments. FIG. 7A is an enlarged plan view of the cassette bay 118 without the surgical cassette 600 attached thereto; FIG. 7B is an enlarged plan view of the cassette bay 118 with the surgical cassette 600 attached thereto. FIGS. 7A-7B are described together herein for clarity.

The cassette bay 118 of the surgical console 100 includes, or is disposed adjacent to, one or more sensors 702 of the sensor system 120 that are configured to detect when a surgical cassette is attached to the cassette bay 118. In the illustrated embodiments, four pairs 704 of optical sensors 702 are shown, wherein two pairs 704 of optical sensors 702 are disposed on each of two lateral sides 710a and 710b of the cassette bay 118. However, other types, numbers, and/or arrangements of sensors 702 are also contemplated. Generally, surgical cassettes for attachment to the cassette bay 118 will have a number of sensing regions corresponding to the number of individual or pairs or sensors 702, as noted above.

Each pair 704 of optical sensors 702 in FIGS. 7A and 7B includes an optical transmitter 702a and an optical receiver 702b. Generally, the optical transmitter 702a in each pair 704 is configured to generate and/or transmit light towards a corresponding target sensing area 706 in the cassette bay 118. The target sensing area 706 coincides to where a sensing region 610 of the surgical cassette 600 is disposed when the surgical cassette 600 is attached to the surgical console 100. Meanwhile, the optical receiver 702b in each pair 704 is configured to receive and detect the light from the corresponding optical transmitter 702a only when the light is reflected by the sensing region 610 of the surgical cassette 600. Accordingly, in certain embodiments, the optical receivers 702b only receive and detect the light when the surgical cassette 600 is attached to the surgical console 100 (as shown in FIG. 7B), and thus, receipt of light by the optical receivers 702b signifies the attachment of the surgical cassette 600. When no surgical cassette 600 is loaded into the cassette bay 118 (as shown in FIG. 7A), the optical receivers 702b in each pair do not receive light from the corresponding optical transmitter 702a, which signifies that a surgical cassette 600 is not attached to the surgical console 100.

The utilization of two or more pairs 704 of sensors 702 for sensing two or more corresponding sensing regions 610 further enables detection by the surgical console 100 of proper alignment of the surgical cassette 600 within the cassette bay 118, which ensures that the surgical cassette 600 is, or will be, securely latched to the cassette bay 118 during use. For example, in the examples of FIGS. 7A and 7B, if the surgical cassette 600 is placed within the cassette bay 118 in an incorrect orientation, at least one of the four sensing regions 610 will not align with their corresponding pair 704 of sensors 702. As a result, only a fraction of the optical receivers 702b will receive light reflected from the surgical cassette 600, thereby indicating to the surgical console 100 that the surgical cassette 600 is misaligned within the cassette bay 118. In certain embodiments, alignment or misalignment of the surgical cassette 600 may be determined by the surgical console 100 using a “majority rule.” That is, if at least a majority (e.g., three of four) of the pairs 704 of sensors 702 detects a corresponding sensing region 610, then the surgical cassette 600 is determined to be aligned within the cassette bay 118; if less than a majority of the pairs 704 of sensors 702 detects a corresponding sensing region 610, then the surgical cassette 600 is determined to be misaligned within the cassette bay 118.

In certain embodiments, the target sensing areas 706 correspond in size and shape to the active areas for the optical receivers 702b. Thus, in some embodiments, the dimensions of the sensing regions 610 of the surgical cassette 600 are based on the dimensions of the target sensing areas 706 and/or active areas of the optical receivers 702b.

In certain embodiments, the optical transmitters 702a each include one or more IR light sources, and the optical receivers 702b each include one or more IR light detectors. In certain embodiments, the optical transmitters 702a each include one or more UV light sources, and the optical receivers 702b each include one or more UV light detectors. In certain embodiments, the optical transmitters 702a each include one or more visible light sources, and the optical receivers 702b each include one or more visible light detectors.

As noted above, the incorporation of the sensing regions 610 facilitates accurate and dependable detection of the surgical cassette 600 when attached to the surgical console 100, particularly where components of the surgical cassette 600 are formed of materials that are transmissive to certain wavelengths of light. Accordingly, by including sensing regions 610 with materials, surface finishes, or coatings that have consistent and invariable light propagation/scattering properties, the surgical cassette 600 can be more consistently detected upon attachment as compared to conventional surgical cassettes, which may have more invariable material and/or surface finish characteristics.

In summary, the present disclosure provides improved ophthalmic surgical cassettes that are able to maintain a more robust hermetic seal with fluidic tubing, and that include sensing features for consistent and accurate detection of the surgical cassettes when attached to surgical consoles.

The previous description is provided to enable any person skilled in the art to practice the various aspects described herein. Various modifications to these aspects will be readily apparent to those skilled in the art, and the generic principles defined herein may be applied to other aspects. Thus, the claims are not intended to be limited to the aspects shown herein, but is to be accorded the full scope consistent with the language of the claims, wherein reference to an element in the singular is not intended to mean “one and only one” unless specifically so stated, but rather “one or more.” Unless specifically stated otherwise, the term “some” refers to one or more. All structural and functional equivalents to the elements of the various aspects described throughout this disclosure that are known or later come to be known to those of ordinary skill in the art are expressly incorporated herein by reference and are intended to be encompassed by the claims. Moreover, nothing disclosed herein is intended to be dedicated to the public regardless of whether such disclosure is explicitly recited in the claims. No claim element is to be construed under the provisions of 35 U.S.C. §112(f) unless the element is expressly recited using the phrase “means for” or, in the case of a method claim, the element is recited using the phrase “step for.”

While various examples of the subject matter have been described above, it should be understood that they have been presented by way of example only, and not by way of limitation. Likewise, the various diagrams may depict an example architectural or other configuration for the disclosure, which is done to aid in understanding the features and functionality that can be included in the disclosure. The disclosure is not restricted to the illustrated example architectures or configurations, but can be implemented using a variety of alternative architectures and configurations. Additionally, although the disclosure is described above in terms of various example examples and aspects, it should be understood that the various features and functionality described in one or more of the individual examples are not limited in their applicability to the particular example with which they are described. They instead can be applied, alone or in some combination, to one or more of the other examples of the disclosure, whether or not such examples are described, and whether or not such features are presented as being a part of a described example. Thus the breadth and scope of the present disclosure should not be limited by any of the above-described examples.

All references cited herein are incorporated herein by reference in their entirety. To the extent publications and patents or patent applications incorporated by reference contradict the disclosure contained in the specification, the specification is intended to supersede and/or take precedence over any such contradictory material.

Unless otherwise defined, all terms (including technical and scientific terms) are to be given their ordinary and customary meaning to a person of ordinary skill in the art, and are not to be limited to a special or customized meaning unless expressly so defined herein.

Terms and phrases used in this application, and variations thereof, especially in the appended claims, unless otherwise expressly stated, should be construed as open ended as opposed to limiting. As examples of the foregoing, the term ‘including’ should be read to mean ‘including, without limitation,’ ‘including but not limited to,’ or the like; the term ‘including’ as used herein is synonymous with ‘including,’ ‘containing,’ or ‘characterized by,’ and is inclusive or open-ended and does not exclude additional, unrecited elements or method steps; the term ‘having’ should be interpreted as ‘having at least;’ the term ‘includes’ should be interpreted as ‘includes but is not limited to;’ the term ‘example’ is used to provide example instances of the item in discussion, not an exhaustive or limiting list thereof; adjectives such as ‘known’, ‘normal’, ‘standard’, and terms of similar meaning should not be construed as limiting the item described to a given time period or to an item available as of a given time, but instead should be read to encompass known, normal, or standard technologies that may be available or known now or at any time in the future; and use of terms like ‘preferably,’ ‘preferred,’ ‘desired,’ or ‘desirable,’ and words of similar meaning should not be understood as implying that certain features are critical, essential, or even important to the structure or function of the disclosed subject matter, but instead as merely intended to highlight alternative or additional features that may or may not be utilized in a particular example of the subject matter. Likewise, a group of items linked with the conjunction ‘and’ should not be read as requiring that each and every one of those items be present in the grouping, but rather should be read as ‘and/or’ unless expressly stated otherwise. Similarly, a group of items linked with the conjunction ‘or’ should not be read as requiring mutual exclusivity among that group, but rather should be read as ‘and/or’ unless expressly stated otherwise.

The term “including as used herein is synonymous with “including,” “containing,” or “characterized by” and is inclusive or open-ended and does not exclude additional, unrecited elements or method steps.

All numbers expressing quantities of ingredients, reaction conditions, and so forth used in the specification are to be understood as being modified in all instances by the term ‘about.’ Accordingly, unless indicated to the contrary, the numerical parameters set forth herein are approximations that may vary depending upon the desired properties sought to be obtained. At the very least, and not as an attempt to limit the application of the doctrine of equivalents to the scope of any claims in any application claiming priority to the present application, each numerical parameter should be construed in light of the number of significant digits and ordinary rounding approaches.

Furthermore, although the foregoing has been described in some detail by way of illustrations and examples for purposes of clarity and understanding, it is apparent to those skilled in the art that certain changes and modifications may be practiced. Therefore, the description and examples should not be construed as limiting the scope of the disclosed to the specific examples and examples described herein, but rather to also cover all modification and alternatives coming with the true scope and spirit of the subject matter.

What is claimed is:

1. A surgical cassette for ophthalmic surgical procedures, the surgical cassette comprising:
a base, comprising:
at least one pump assembly disposed in the base;
a plurality of first channels in fluid communication with the at least one pump assembly, wherein one or more of the plurality of first channels are in fluid communication with a source of pressure or vacuum; and
one or more valve assemblies disposed in the base and configured to control fluid communication between the plurality of first channels of the base; and
a tubing adapter coupled to the base, the tubing adapter comprising a plurality of ports in fluid communication with at least one of the plurality of first channels disposed in the base, each of the plurality of ports configured to receive a corresponding fluidic tube for coupling the fluidic tube to the surgical cassette.

2. The surgical cassette of claim 1, wherein:
the plurality of ports comprises a first port for aspiration of fluids into the surgical cassette, a second port for irrigation of fluids from the surgical cassette, and a third port for receiving the fluids for irrigation into the surgical cassette.

3. The surgical cassette of claim 1, wherein each of the plurality of ports comprises:
a first portion at a proximal end of the tubing adapter and configured to engage with a corresponding connector of the base to couple the tubing adapter to the base; and
a second portion at a distal end of the tubing adapter and configured to receive the corresponding fluidic tube.

4. The surgical cassette of claim 3, wherein each of the plurality of ports comprises:
a second channel extending between the proximal end and the distal end of the tubing adapter, the second channel comprising:
a first segment disposed through the first portion and having a first lateral dimension; and
a second segment disposed through the second portion and having a second lateral dimension, wherein the first lateral dimension and the second lateral dimension of the second channel facilitate a stable fluid flow path for consistent fluid flow speed and pressure during use of the surgical cassette.

5. The surgical cassette of claim 1, wherein:
the base is formed of a first material; and
the tubing adapter is formed of a second material.

6. The surgical cassette of claim 5, wherein the second material comprises at least one of polycarbonate, polypropylene, polyethylene, polyvinyl chloride, or polystyrene.

7. The surgical cassette of claim 1, wherein:
the tubing adapter is welded to a dock of the base to secure the tubing adapter to the base.

8. The surgical cassette of claim 7, wherein each of the ports is bonded to the corresponding fluidic tube via a bonding agent.

9. The surgical cassette of claim 1, further comprising:
one or more sensing regions for facilitating detection of the surgical cassette when the surgical cassette is attached to a surgical console.

10. The surgical cassette of claim 9, wherein:
the one or more sensing regions comprise at least one of a material, a surface finish, or a coating that facilitates detection of the surgical cassette with an optical sensor of the surgical console by reducing scattering of target wavelengths of light, reducing transmission of the target wavelengths of light through the surgical cassette, or increasing reflection of the target wavelengths of light.

11. A surgical cassette for ophthalmic surgical procedures, the surgical cassette comprising:
a base, comprising:
at least one pump assembly disposed in the base;
a plurality of first channels in fluid communication with the at least one pump assembly, wherein one or more of the first channels is in fluid communication with a source of pressure or vacuum;
one or more valve assemblies disposed in the base and configured to control fluid communication between the plurality of first channels of the base; and
one or more sensing regions disposed on an external surface of the base for facilitating detection of the surgical cassette when the surgical cassette is attached to a surgical console; and
a cover coupled to the base, the cover comprising at least one port in fluid communication with at least one of the plurality of first channels disposed in the base.

12. The surgical cassette of claim 11, wherein:
the one or more sensing regions comprise at least one of a material, a surface finish, or a coating that facilitates detection of the surgical cassette with an optical sensor of the surgical console by reducing scattering of target wavelengths of light, reducing transmission of the target wavelengths of light through the surgical cassette, or increasing reflection of the target wavelengths of light.

13. The surgical cassette of claim 12, wherein the one or more sensing regions comprise a polished surface finish with a Society of the Plastic Industry (SPI) surface finish grade of SPI A1, SPI A2, or SPI A3.

14. The surgical cassette of claim 12, wherein the one or more sensing regions comprise at least one of:
a metallic paint;
a plated metal layer;
an anodized layer;
a pad printed with reflective ink;
a reflective hot stamped foil;
an in-mold foil decoration (IMD); or
a reflective label.

15. The surgical cassette of claim 12, wherein the target wavelengths comprise wavelengths of infrared (IR) light between 780 nm (nanometers) and 1 mm (millimeters).
